(19) United States
(12) Patent Application Publication
(10) Pub. No.: US 2005/0042298 A1
(43) Pub. Date: Feb. 24, 2005

(54) Immunonanoparticles

(76) Inventors: William M. Pardridge, Pacific Palisades, CA (US); Jean-Christophe Olivier, Poitiers (FR)

Correspondence Address: DAVID J. OLDENKAMP, ESQ., SHAPIRO & DUPONT LLP, 233 WILSHIRE BOULEVARD, SUITE 700, SANTA MONICA, CA, 90401, US

(21) Appl. No.: 10/647,197

(22) Filed: Aug. 20, 2003

(51) Int. Cl. A61K 9/14

(52) U.S. Cl. 424/489

(57) ABSTRACT

A functionalized composition for use in forming an immunonanoparticle. The functionalized composition includes a nanoparticle-forming polymer and a polymeric strand having a first end attached to the nanoparticle-forming polymer and a second end to which a conjugation agent is attached. The functionalized composition is combined with non-functionalized compositions to form nanoparticles compositions that can be used to provide targeted delivery of drugs and diagnostic agents.

### BACKGROUND OF THE INVENTION

1. Field of the Invention

The present invention relates generally to compositions that are used to provide targeted delivery of drugs and diagnostic agents. More particularly, the present invention is directed to nanoparticle compositions referred to as “immunonanoparticles” that may be used to provide targeted delivery of a wide variety of payloads including small molecules, proteins, drugs and diagnostic agents. The invention is also directed to the compositions that are used in the preparation of such immunonanoparticles.

2. Description of Related Art

The publications and other reference materials referred to herein to describe the background of the invention and to provide additional detail regarding its practice are hereby incorporated by reference. For convenience, the reference materials are grouped in the appended bibliography.

Targeted drug delivery using nanocontainers, such as liposomes or nanoparticles, requires that the nanocontainer be delivered to the target organ in parallel with restricted uptake by non-targeted tissues. Liposomes or nanoparticles are rapidly removed from the bloodstream by cells lining the reticulo-endothelial (RES) system, and this greatly reduces the area under the plasma concentration curve (AUC) and pharmacological effect. The rapid RES uptake can be reduced, and the plasma AUC increased, by conjugating to the surface of the liposome several thousand polymeric strands, such as polyethyleneglycol (PEG) (1). Pegylated liposomes are not specifically targeted to tissues, but this can be accomplished by tethering to the tips of 1-2% of the PEG strands a targeting ligand, such as a receptor-specific monoclonal antibody. The pegylated immunoliposome (PIL) can be delivered across microvascular barriers by receptor-mediated transcytosis (2).

Nanoparticles have certain advantages relative to liposomes since nanoparticles can be freeze-dried for long-term storage. Nanoparticles are prepared from polymeric precursors such as poly(lactic acid) (PLA) homopolymer or poly(lactic-co-glycolic acid) (PLGA) heteropolymer. PLA and PLGA are biocompatible and biodegradable polyesters (3). Similar to liposomes, nanoparticles are rapidly removed from the blood by the RES, and this uptake is inhibited by conjugation of PEG to the nanoparticle surface (4-6). Pegylated nanoparticles (PN) are formed by a hydrophobic PLA core which is surrounded by a hydrophilic PEG “corona” layer (5). These can be freeze-dried (4) and loaded with small molecules (7), proteins (8) or plasmid DNA (9). Nanoparticles made of methoxyPEG₂₀₀₀-PLA₃₀₀₀₀ with respective molecular weights of 2000 and 30,000 Daltons for the methoxyPEG and PLA moieties, were shown to have an acceptable safety profile in rats (10). Pegylated PLA nanoparticles have a prolonged plasma half-life (t_{1/2}=6h) in comparison with PLA nanoparticles coated with albumin or with poloxamer F68 (t_{1/2}=2-3 min) (6).

Pegylated nanoparticles (PN), like pegylated liposomes, are not specifically targeted to tissues in vivo, as this requires the attachment of a targeting ligand to the tip of the PEG strands (2). Targeting ligands, such as endogenous peptides or peptidomimetic MAb's, can be conjugated to the PEG strands of preformed PN's, if a fraction of the PEG strands contains a terminal reactive group. Yasugi et al. (11) synthesized sugar-terminated PEG-PLA block copolymers, using a multi-step method that was not adapted to the conjugation of protein ligands, such as antibodies. Most targeting ligands are peptide or protein macromolecules, and mild reaction conditions are necessary to preserve their biological and receptor binding activity. Pegylated immunoliposomes (PIL) are prepared by conjugating thiolated antibodies to pegylated liposomes, wherein a small fraction of the PEG polymer is comprised of a bifunctional PEG, that contains a lipid group such as distearoylphosphatidylethanolamine (DSPE) at one terminus and a reactive maleimide moiety at the other terminus (2). The maleimide function reacts with thiolated proteins in conditions that maintain their biological and binding properties.

### SUMMARY OF THE INVENTION

The present invention is based on the discovery of special functionalized compositions that can be used to form nanoparticle compositions (immunonanoparticles) that are useful as a delivery system for a variety of payloads including drugs and diagnostic agents. The functionalized compositions include a polymeric strand that has a nanoparticle-forming polymer attached to one end and a conjugation agent attached to the other end. The conjugation agent is capable of binding to targeting agents, such as endogenous peptides or peptidomimetic monoclonal antibodies (MAb).

The functionalized compositions can be used with other nanoparticle-forming entities to form immunonanoparticles. In one aspect of the invention, the functionalized compositions are combined with non-functionalized compositions to form immunonanoparticles. The non-functionalized compositions include a nanoparticle-forming polymer and a polymeric strand without the conjugation agent. The functionalized and non-functionalized compositions are combined together to form a nanoparticle composition having a core that is surrounded by a corona of polymeric strands with conjugation agents being attached to some of the strands. Targeting agents are attached to the conjugation agents to provide targeting of the nanoparticle composition to desired receptor sites. The desired drug or diagnostic payload, if any, is incorporated into the polymeric core of the nanoparticle composition during formation of the core.

As one feature of the present invention, it was discovered that immunonanoparticles could be formed such that the conjugation agents and attached targeting agents protrude above the corona that surrounds the particle core. This is accomplished by combining non-functionalized compositions having relatively short polymeric strands with functionalized compositions having relatively long polymeric strands. The strands of the non-functionalized compositions form the body of the protective corona around the nanoparticle while the strands of the functionalized compositions extend above the corona to provide enhanced exposure of the conjugation agent for binding with various targeting agents.

The above described and many other features and attendant advantages of the present invention will become better understood by reference to the following detailed description when taken in conjunction with the accompanying drawings

### BRIEF DESCRIPTION OF THE DRAWINGS

FIG. 1 is a diagrammatic representation showing the step of combining functionalized and non-functionalized compositions together in accordance with a preferred exemplary embodiment of the present invention to provide a nanoparticle composition that includes a core composed of nanoparticle-forming polymers (NPFP) and a drug or diagnostic agent plus a corona surrounding the core. The corona includes polymeric short strands (PSS) and polymeric long strands (PSL) and conjugation agents (CA) attached to some of the strands.

FIG. 2 is a diagrammatic representation of a nanoparticle composition (immunonanoparticle) in accordance with the present invention that further includes targeting agents (TA) that are attached to the conjugation agents.

FIG. 3(A) is a schematic diagram of bi-functional PEG₃₅₀₀ containing a free hydroxyl group at one terminus, for conjugation to the PLA, and a maleimide moiety at the other terminus, for conjugation to a thiolated targeting ligand. PEG₃₅₀₀=polyethyleneglycol of 3500 Daltons molecular weight. (B) is a diagram of a pegylated immunonanoparticle showing drug encapsulated within an 100-150 nm nanoparticle containing a hydrophilic corona of several thousand strands of PEG. About 1-2% of the PEG strands are conjugated with a receptor (R)-specific targeting monoclonal antibody (MAb).

FIG. 4(A) shows the formula and ¹H NMR spectrum of methoxyPEG₂₆₀₀-PLA₄₀₀₀₀ copolymers in deuterochloroform at 300.0K. (B) shows the formula and ¹H NMR spectrum of maleimide-PEG₃₅₀₀-PLA₄₀₀₀₀ block copolymers in deuterochloroform at 300.0K.

FIG. 5 is a transmission electron micrograph of pegylated nanoparticles negatively stained with phosphotungstic acid solution. The magnification bar in lower right hand comer is 120 nm.

FIG. 6 shows the elution profile of [³H]-OX26 MAb-conjugated pegylated immunonanoparticles obtained by Sepharose CL-4B gel filtration chromatography. Fractions 8 to 11 correspond to [³H]-OX26 MAb-conjugated PINs and fractions 17 to 32 to unconjugated [³H]-OX26 MAb.

FIG. 7 is a transmission electron micrograph of pegylated immunonanoparticles negatively stained with phosphotungstic acid solution. The OX26 antibodies conjugated to the pegylated nanoparticles are revealed by binding with a conjugate of 10 nm gold and an anti-mouse IgG secondary antibody. The magnification bar is 15 nm.

### DETAILED DESCRIPTION OF THE INVENTION

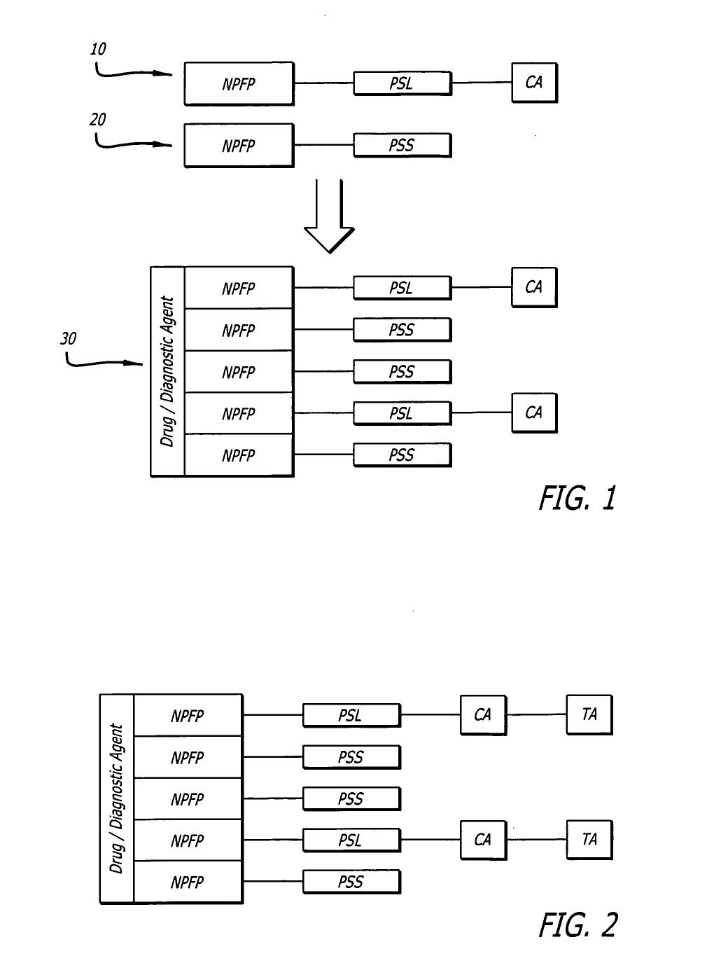

A functionalized composition in accordance with the present invention is shown diagrammatically in FIG. 1 at 10. The functionalized composition includes a nanoparticle-forming polymer (NPFP), a polymeric strand (PSL) and a conjugation agent (CA). The functionalized composition may be used alone or in combination with a variety of other entities to form immunonanoparticles. For the purposes of this specification, “immunonanoparticles” include any particle that has a diameter of 1000 nanometers or less and which includes a targeting agent (TA) that is capable of selectively binding to a biological moiety, such as an antigen or cellular receptor. The immunoparticle may or may not include a payload, such as a drug or diagnostic agent. Preferred immunonanoparticles have diameters that are less than 300 nanometers. Particularly preferred immunonanoparticles have diameters that are in the range of 50 to 150 nanometers.

In a preferred exemplary embodiment as shown diagrammatically in FIG. 1, the functionalized composition 10 is combined with non-functionalized compositions 20 and a suitable drug or diagnostic agent to form a nanoparticle compositions 30. The non-functionalized composition also includes a nanoparticle-forming polymer (NPFP) and a polymeric short strand (PSS). The non-functionalized composition does not include a conjugation agent. The PSS polymeric strand of the non-functionalized composition is preferably shorter than the polymeric long strand (PSL) of the functionalized composition. The two compositions and drug or diagnostic agent are combined according to well-known polymerization procedures to form the immunonanoparticle 30. Although the use of a non-functionalized composition of the type shown at 20 is preferred, other non-functionalized nanoparticle forming entities may be combined with the functionalized composition to form the immunonanoparticle. For example, all or a portion of the non-functionalized composition may include a NPFP without an attached polymeric strand or other types of corona forming ligands can be attached to the NPFP.

The final immunonanoparticle, as shown diagrammatically in FIG. 2, is formed by treating the nanoparticle composition 30 with a targeting agent. If desired, the targeting agent can be attached to the functionalized composition 10 before it is combined with the non-functionalized composition 20.

The PSL portion of the functionalized composition 20 is preferably polyethylene glycol (PEG). Other suitable PSL portions include polymer strand forming materials such as sphingomyelin. The molecular weight of the polymer strand (PSL) should be such that the conjugation agent and targeting agent can be attached to the outer (second) end of the PSL without interfering with formation of the immunonanoparticle and such that the targeting agent is sufficiently distant from the nanoparticle core that it is able to bind or otherwise react with the intended target moiety or site. For PEG polymeric strands, preferred molecular weights range from about 1,000 to 50,000 daltons. A particularly preferred molecular weight range for a PEG PSL is between 3,000 and 20,000 daltons. The PSL must be bifunctional in that is must have a first end that is attachable to the NPFP and a second end that is attachable to a conjugation agent. This is preferably accomplished by providing a free hydroxy group at the first end of the PSL which allows conjugation with the NPFP. A methoxy or other similar group is preferably located at the second end of the PSS. The CA is located at the second end of the PSL. The particular chemistry that is used to provide bifunctionality to the PSL in not particularly important provided that the NPFP is attached to one end and the CA to the other without adversely affecting the ability of the NPFP to form polymeric nanoparticles and without adversely affecting the ability of the CA to bind to targeting agents (TA).

Poly(lactic acid) is a preferred NPFP. Other suitable NPFP's include poly(glycolide), poly(lactide-co-glycolide), polybutylcyanocrylate (PBCA), poly(ε-caprolactone) (PCL), monomethoxypoly(ethylene oxide)-poly(lactic acid) (MPEO-PLA), hexadecylcyanoacrylate (PHDCA). The molecular weight of the NPFP should be such that, when polymerized, the NPFP is capable of forming nanoparticles having sizes of less that 1000 nanometers and preferable in the range of 50 to 150 nanometers. Preferred poly(lactic acid) for use as the NPFD will have molecular weights of between about 20,000 and 60,000 daltons. Molecular weights on the order of 40,000 daltons are particularly preferred. The NPFP is attached to the PSL using known conjugation techniques that will vary depending upon the particular NPFP and PSL.

A wide variety of conjugation agents may be attached to the second end of the polymeric strand (PSL). Maleimide is a preferred conjugation agent, as this forms a stable thio-ether linkage with thiolated TA. Other suitable conjugation agents include a carboxyl moiety, as this forms an amide linkage with an amino group on the TA, an N-hydroxy succinimide ester, as this forms an amide linkage with an amino group on the TA, a hydrazide group, as this forms a linkage with a carboxyl group on the TA.

Non-functionalized compositions 20 are basically the same as the functionalized compositions described above except that they do not include a conjugation agent. In addition, it is preferred that the polymeric strand of the non-functionalized compositions (PSS) be shorter than the PSL of the functionalized compositions. The non-functionalized compositions are made in the same manner as the functionalized compositions except that the conjugation agent is not substituted or otherwise added to the second end of the PSS. The second end is preferably terminated with a methoxy group. However, it may be terminated with any suitable end group. The non-functionalized composition may be made from the same NPFP and polymer strand materials as the functionalized composition. For a particular immunonanoparticle, the NPFP of the non-functionalized and functionalized compositions may be the same or they may be different. In addition, the polymers used to form the polymeric strands may be the same or different. Preferably, the NPFP and polymeric strand materials will be the same for both the functionalized and non-functionalized compositions. Poly(lactic acid) is a preferred NPFP and PEG is a preferred PSS for non-functionalized compositions.

The length of the PSL and PSS polymeric strands may be the same or they may be different. It is preferred that the PSL polymeric strand be longer than the PSS strand in order to make the CA and attached TA more accessible to the target. The difference in length between the PLS and PSS will depend upon the particular CA and TA that is used for a particular immunonanoparticle. The difference is length should be chosen so that the TA is exposed above the corona surface sufficiently to allow target binding. For PEG polymeric strands, it is preferred that the PSS have a molecular weight that is between 200 and 2000 daltons less than the molecular weight of the PSL.

The relative amounts of non-functionalized and functionalized compositions that are combined to form the immunonanoparticle may be varied depending upon a number of factors including the binding strength of the TA with the target as well as the size and chemical reactivity of the various elements of the compositions. In general, the amount of functionalized composition in the immunonanoparticle will be substantially less that the amount of non-functionalized composition. The ratio of functionalized composition to non-functional composition will typically be 1:30. Preferably, the ratio of functionalized composition to non-functionalized composition will be between 1:5 and 1:100.

The functionalized 10 and non-functionalized 20 compositions are preferably combined together as shown in FIG. 1 to form a nanoparticle composition 30. The desired drug or diagnostic agent, if any, is incorporated into the nanoparticle core during formation of the nanoparticle composition 30 as is well known in the art. The resulting nanoparticle composition is ready for combination with a targeting agent and production of drug encapsulated pegylated nanoparticles. The drug/pegylated nanoparticle containing the attached targeting agent (TA) is shown in FIG. 2. However, if desired, the targeting agent can be attached to the CA on the functionalized composition prior to combination with the non-functionalized composition. In this way, the resulting nanoparticle already has the targeting agent in place when the nanoparticle composition, with or without the incorporated drug or diagnostic agent, is formed. This embodiment of the invention requires that the TA is of sufficient composition to retain biological activity despite treatment with the emulsion or related procedures required to form the nanoparticles.

The method used for combining the functionalized and non-functionalized compositions to form immunonanoparticles can be any of the conventional polymerization procedures that are well known for forming copolymers including solution polymerization and the like. The preferred method for formation of the nanoparticles is the emulsion/solvent evaporation method.

The polymeric core of the immunonanoparticle can be loaded with a wide variety of payloads including drugs and diagnostic agents. The procedures for loading polymeric nanoparticles with such small payloads are well known. The preferred method is to load the drug with the polymers and form the nanoparticles with the emulsion/solvent evaporation method. Exemplary drugs include small molecules, peptides, recombinant proteins, antisense agents and polymers, and plasmid DNA. Exemplary diagnostic agents include peptide or antisense radiopharmaceuticals or magnetopharmaceuticals, or small molecule radiopharmaceuticals or small molecule magnetopharmaceuticals.

The immunonanoparticles in accordance with the present invention may be used in vivo as a drug or diagnostic agent or they may be used in vitro for diagnostic or research purposes. When used in vivo, the immunonanoparticles may be combined with any suitable pharmaceutical carrier for intravenous administration. Intravenous administration is the preferred route since it is the least invasive. Other routes of administration are possible, if desired. Suitable pharmaceutically acceptable carries include saline, Tris buffer, phosphate buffer or any other aqueous solution typically used as a carrier for drugs and diagnostic agents.

A therapeutically effective amount of the immunonanoparticles will vary widely depending upon the individual being treated and the particular drug being administered. The appropriate dose will be established by procedures well known to those of ordinary skill in the art.

The following examples describe the preparation and use of functionalized compositions in accordance with the present invention. The functionalized compositions include a series of copolymers composed of a poly(α-hydroxy acid) polymer block chain covalently linked to a poly(ethylene glycol) (PEG) terminated with an active maleimide function (i.e., maleimide-poly(ethylene glycol)-block-poly(α-hydroxy acid) copolymers). The copolymers can be used to prepare pegylated nanoparticles (PN), microspheres or any other device for biomedical applications and permits the conjugation of thiolated ligands. The ligands include endogenous peptides or peptidomimetic monoclonal antibodies (MAb) that bind to specific receptors in vivo and trigger transfer of the nanoparticle across biological barriers using endogenous receptor-mediated transcytosis and endocytosis systems. The conjugation of a receptor-specific MAb to the PN results in the formation of a pegylated immunonanoparticle (PIN).

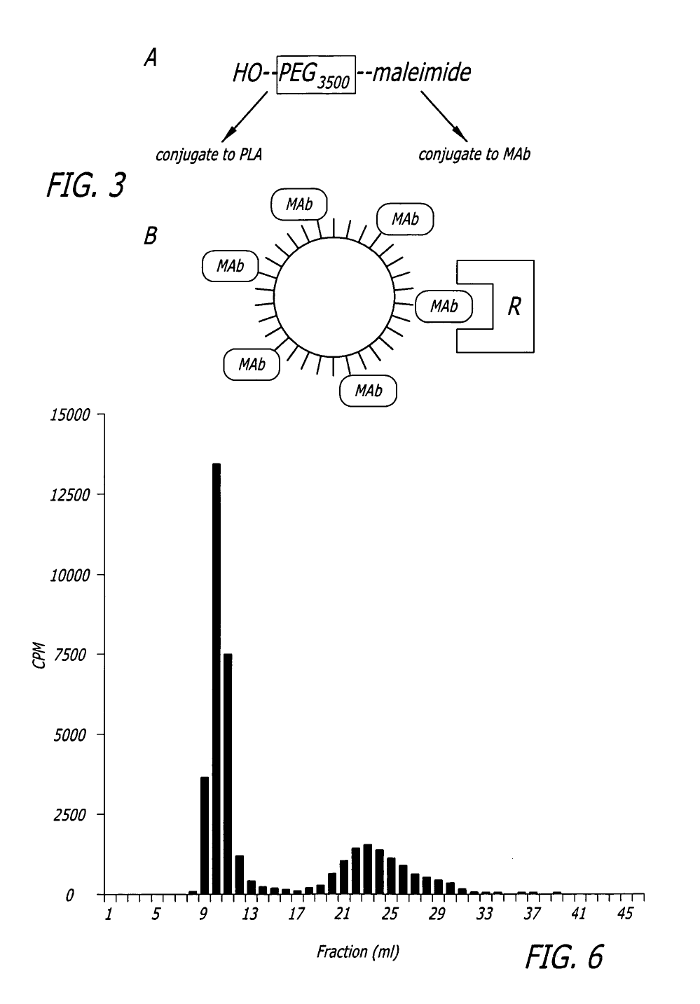

The maleimide-PEG-PLA copolymers described in the following examples possess a terminal chemically active maleimide function, which is synthesized from the starting monomer and the bifunctional PEG moiety shown in FIG. 3A, which has a free hydroxyl group at one end, for covalent attachment to the PLA polymer, and a maleimide group at the other end for conjugation to the thiolated ligand. Once included in the structure of nanoparticles or any other device, the maleimide-PEG moiety of maleimide-PEG-PLA is exposed in aqueous solution and the terminal chemically active maleimide function is available for reacting with thiol groups on the TA. The maleimide function can react under mild conditions with thiol groups to form a covalent linkage between the molecules to be conjugated and the PEG-coated device.

The synthesis of PLA and PGA homopolymers and of PLGA copolymers is based on the ring opening polymerization of lactide or/and glycolide in the presence of a catalyst (generally stannous octoate) under moisture-free conditions in the presence of an organic solvent (toluene, xylene) (25). To prepare methoxy-PEG-poly(α-hydroxy)acids, the same procedures are used (12, 13 and 26). In this case, the hydroxyl end group of methoxy end-capped PEG initiates the polymerization by a nucleophilic attack of the lactide or glycolide cyclic dimers and is covalently linked to the PLA chain in formation. The length of the poly(α-hydroxy)acid block depends on the ratio between the amount of cyclic dimers and the amount of initiating methoxy end-capped PEG in the reaction medium. The reaction yield is generally high. Maleimide-PEG-PLA is prepared using the same method with maleimide-PEG and lactide cyclic dimers as starting material. The hydroxyl end group of maleimide-PEG initiates the polymerization of lactide cyclic dimers.

The following examples set forth the synthesis of pegylated immuno-nanoparticles (PIN). The PIN synthesis included the initial synthesis of a copolymer composed of maleimide-PEG₃₅₀₀-PLA₄₀₀₀₀, which is produced from a bifunctional PEG₃₅₀₀ containing a maleimide moiety at one terminus and a free hydroxyl group at the other terminus (FIG. 3A). The maleimide-PEG₃₅₀₀-PLA₄₀₀₀₀ copolymer was blended with a non-functionalized composition (methoxyPEG₂₆₀₀-PLA₄₀₀₀₀) to prepare pegylated nanoparticles, which were then conjugated to a thiolated targeting MAb in order to obtain the final PIN product.

The following examples of the synthesis of maleimide-PEG-block-poly(lactide) (PLA) functionalized compositions is limited to only one type of maleimide-poly(ethylene glycol)-block-poly(α-hydroxy acid) copolymers. It will be understood by those skilled in the art that the present invention and the synthesis procedures also cover any cyclized (α-hydroxy)acids (Lactide, glycolide) and a wide range of copolymers made of poly(lactic acid), poly(glycolic acid) or poly(lactic-co-glycolic acid) of various molecular weights linked to PEG-maleimide (maleimide-PEG-block-poly(α-hydroxy)acid copolymers of various molecular weights.

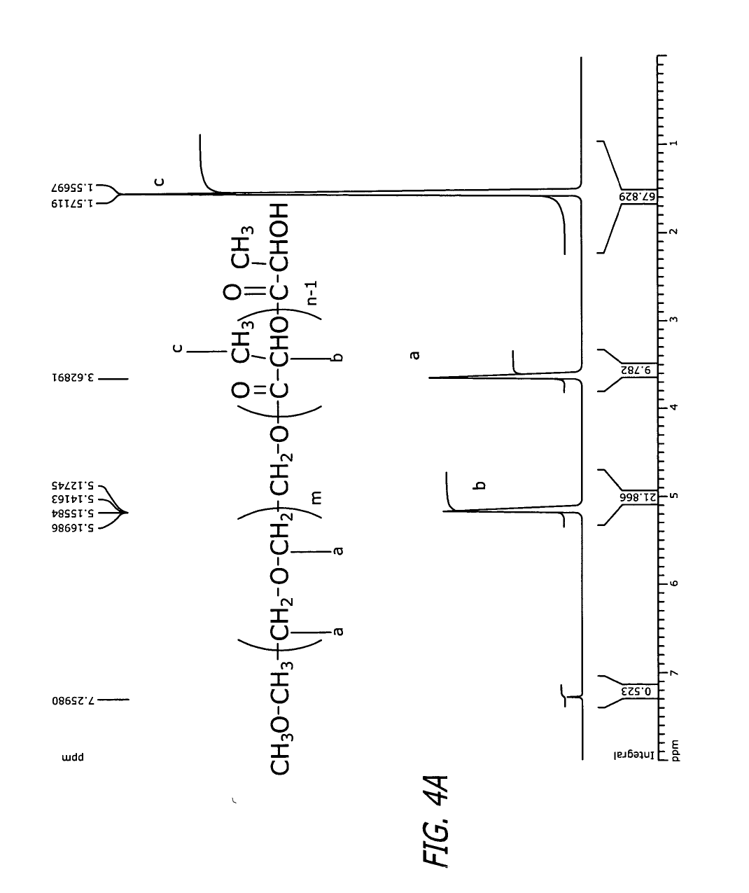

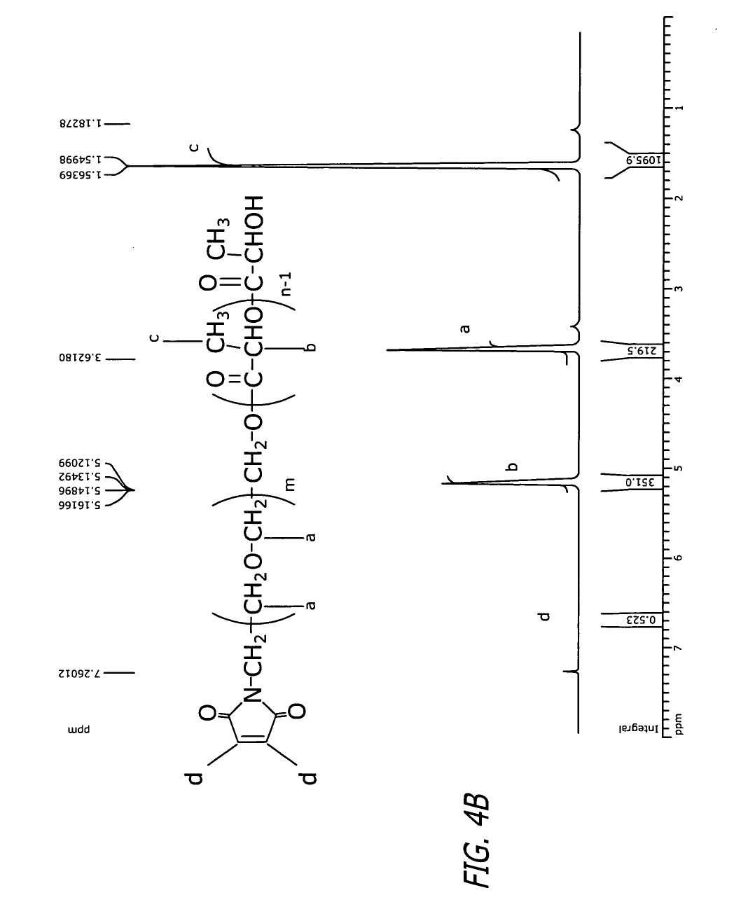

A preferred exemplary functionalized composition in accordance with the present invention is the maleimide-poly(ethylene glycol)-block-poly(lactide) (maleimide-PEG-PLA) shown in FIG. 4B. For any of the proposed applications, the copolymer is preferably be blended with a non-functionalized composition, such as poly(lactic acid), poly(glycolide) (PGA) or poly(lactide-co-glycolide) (PLGA) and/or copolymers of PLA, PGA or PLGA linked to methoxy-PEG. An example of a non-functionalized composition is PEG of 3500 Daltons linked to 40,000 Dalton PLA as shown in FIG. 4A. In the case of nanoparticles or of microspheres, the PLA moieties will constitute the core of the nanoparticles and the PEG moiety the hydrophilic layer covering the surface of the nanoparticle core (FIG. 3B). This ‘corona’ of PEG minimizes the rapid uptake of the nanoparticle by the reticulo-endothelial system (RES) (27). The maleimide-PEG-PLA copolymer, anchored in the core of the nanoparticles through the PLA moiety has its maleimide function protruding out of the PEG layer that surrounds the nanoparticles and is available to conjugate to any kind of thiolated molecules (protein, glycoprotein or others). Such molecules are activated by a thiolating reagent (2-iminothiolane or Traut's reagent, for example). Under mild conditions, compatible with the retention of the biomolecules' activities, the maleimide function will react with thiol groups of these thiolated biomolecules, to form a covalent linkage between the nanoparticles and the biomolecules. An exemplary application is the use of immunonanoparticles to deliver drugs (or genes) to specific target sites via special receptor systems expressed at the target site (FIG. 3B). The same procedure can be applied to any device containing this novel copolymer. Therefore, maleimide-PEG-PLA can have numerous applications for drug targeting, for diagnostic purpose, and for any other composition made of PLA, PGA or PLGA that needs to have its surface modified by covalent conjugation of a particular molecule.

Materials. Ethyl acetate (99.5% pure) and stannous octoate (95% pure) were obtained from Sigma Chemical Co. (St. Louis, Mo.). Toluene (99.8% pure), dichloromethane (>99.9% pure), ethyl ether (98% pure), calcium hydride (95% pure), L-lactide (98% pure) were purchased from Aldrich (Milwaukee, Wis.). Methoxypolyethyleneglycol, Mn=2600 Daltons (MethoxyPEG₂₆₀₀) was obtained from Shearwater Polymers, Inc. (Huntsville, Ala.). Hydroxy-polyethyleneglycol-maleimide (FIG. 3A), Mn=3501 (Maleimide-PEG₃₅₀₀) was custom-synthesized by Shearwater Polymers, Inc. The number average molecular weights (Mn) are per the manufacturer's specifications. Anti-mouse IgG immunoglobulin gold (10 nm) conjugate (1.4×10¹³ particles/ml) was obtained from Sigma. Ethyl acetate and toluene were dried and distilled under argon over calcium hydride. L-lactide was dried under vacuum (180 mm Hg) at 70° C. for 30 min, re-crystallized from dry ethyl acetate, and then dried at 70° C. under an argon flow. Stannous octoate was distilled under vacuum, the fraction distilling at 160° C. and 30 mm Hg corresponding to the purified compound as assessed by ¹H-NMR. MethoxyPEG₂₆₀₀ was dried under vacuum and under magnetic stirring at 70° C. for 30 min. Maleimide-PEG₃₅₀₀ was dried under vacuum at room temperature for 30 min.

Copolymer synthesis and characterization. The methoxypolyethyleneglycol₂₆₀₀-poly(lactic acid)₄₀₀₀₀, designated methoxyPEG₂₆₀₀-PLA₄₀₀₀₀ copolymer (non-functionalized composition) and the maleimide-polyethyleneglycol₃₅₀₀-poly(lactic acid)₄₀₀₀₀, designated maleimide-PEG₃₅₀₀-PLA₄₀₀₀₀ (functionalized composition) were synthesized by ring opening polymerization in dry toluene under moisture-free high-purity argon atmosphere, according to a method adapted from Bazile et al. (12). For the methoxyPEG₂₆₀₀-PLA₄₀₀₀₀ synthesis, 3.75 g recrystallized L-lactide was combined with 208 mg methoxyPEG₂₆₀₀ and 20 mg stannous octoate, as solutions in dry toluene (respective concentrations: 62.5 mg/ml and 20 mg/ml). For the maleimide-PEG₃₅₀₀-PLA₄₀₀₀₀ synthesis, 3.75 g purified L-lactide was combined with 294 mg maleimide-PEG₃₅₀₀ and 20 mg stannous octoate, as solutions in dry toluene (respective concentrations: 150 mg/ml and 20 mg/ml). In both cases, the final volume of dry toluene was 10 ml. The polymerization was then carried out under magnetic stirring and under moisture-free high-purity argon atmosphere at 110° C. for 2 h. At the end of the polymerization step, the copolymers were purified and recovered as follows. To the reaction medium, slightly cooled dichloromethane (10 ml) was added. The copolymers were then precipitated by addition of 30 ml ethyl ether and recovered on a Buchner funnel with a fritted disk. They were re-dissolved in 20 ml dichloromethane, precipitated by 20 ml ethyl ether and recovered on the same filter. They were then dried at 70° C. under an argon flow for 30 min, and under vacuum at 70° C. for 1 h. The two copolymers were analyzed by ¹H and ¹³C NMR spectroscopy at 300.0K using a Brucker Advance 500 spectrometer operating at 500 MHz and with deuterochloroform as the solvent. Chemical shifts in ppm (δ) were determined using the chloroform signals at 7.26 ppm (¹H) or at 77.00 ppm (¹³C) as references. The integrals of the peaks corresponding to the PLA methylene protons (δ 5.1 ppm) and the PEG methylene protons (δ 3.6 ppm) were used to determine the weight ratio of PLA to PEG and to calculate the average number molecular weight (Mn) of the PLA moiety (13). The integral of the peak corresponding to the maleimide protons (δ 6.7 ppm) was used to check for the preservation of the maleimide function in the synthesized maleimide-PEG₃₅₀₀-PLA₄₀₀₀₀.

Pegylated nanoparticle preparation. Nanoparticles were made of a blend of methoxyPEG₂₆₀₀-PLA40000 and maleimide-PEG₃₅₀₀-PLA₄₀₀₀₀. They were prepared using the emulsion/solvent evaporation technique (14). Fifty μl of water (aqueous inner phase) was emulsified by sonication (30s) using a probe sonicator (Branson sonifier cell disruptor) at the maximum power in 1 ml of a solution of methoxyPEG₂₆₀₀-PLA₄₀₀₀₀ (24.2 mg/ml) and maleimide-PEG₃₅₀₀-PLA₄₀₀₀₀ (0.8 mg/ml) in dichloromethane (intermediate organic phase). This primary emulsion was then emulsified by sonication (30s) in 2 ml of a 1% sodium cholate aqueous solution (aqueous outer phase). The w/o/w emulsion thus obtained was diluted into 38 ml of a 0.5% sodium cholate aqueous solution under rapid magnetic stirring. After 1 min, dichloromethane was evaporated at low pressure and at 40° C. using a Büchi R-3000 rotary evaporator (rotation speed set at 90% maximum speed). Nanoparticles were then centrifuged at 45000 g (using a J2-21 Beckman Centrifuge equipped with a JA-20 rotor) and, after discarding the supernatant, they were resuspended in 0.5 ml of a 0.01M HEPES buffer pH 7 containing 0.15M NaCl and 0.1 mM EDTA. The mean (number based) diameter of the nanoparticles was determined by quasi-elastic light scattering (QELS) using a Microtrac Ultra Particle Analyzer (Leeds-Northrup, St. Petersburg, Fla.). Nanoparticles were also observed by transmission electron microscopy. Nanoparticle concentration was determined by turbidimetry.

Pegylated immunonanoparticle preparation The anti-rat transferrin receptor OX26 MAb was harvested from serum-free OX26 hybridoma-conditioned media using routine methods and purified by protein G Sepharose affinity chromatography. OX26 MAb was radiolabelled with N-succinimidyl[2,3-³H]propionate using methods well known. The [³H]-OX26 MAb had a specific activity of 0.12 mCi/mg and a TCA precipitability of 95%. OX26 MAb (1 mg, 6.65 nmol) supplemented with [³H]-OX26 MAb (1 μCi) was thiolated by reacting for 60 min with a 40:1 molar excess of 2-iminothiolane (Traut's reagent) in 0.1 5M sodium borate buffer, pH=8 supplemented with 0.1 mM EDTA. The buffer was exchanged with 0.01M HEPES pH=7 containing 0.15M NaCl and 0.1 mM EDTA using a Centricon YM-30 concentrator tube (Amicon) and thiolated OX26 MAb was concentrated to a volume of 50 μl. Thiolated OX26 MAb was then mixed with nanoparticles at a thiolated OX26 MAb:maleimide molar ratio of 1:3. The volume of the mixture was 1 ml and the conjugation of the MAb to the pegylated nanoparticle was performed overnight on a rotating plate set at a low speed. The reaction mix was then applied to a 1.5×20 cm Sepharose CL-4B column and was eluted with 0.05M HEPES buffer pH=7. To determine the OX26 MAb concentration in each collected fraction, aliquots (100 μl) of the column eluate were analyzed by scintillation counting using a Tricarb 2100TR Liquid Scintillation Analyzer (Packard Instrument Co., Downers Grove, Ill.). The PIN fractions were milky and were visually identified and collected, and the nanoparticle concentration was determined by turbidimetry. The average number of OX26 MAb molecules conjugated per nanoparticle was calculated by dividing the number of OX26 MAb's in each fraction by the calculated average number (n) of nanoparticles using the following equation:
n=6m/(Π×D³×ρ)
where m is the nanoparticle weight, D the number based mean nanoparticle diameter determined by quasielastic light scattering (QELS), ρ the nanoparticle weight per volume unit (density), estimated to be 1.1 g/cm³.

Electron microscopy of gold-labeled pegylated immunonanoparticles. PIN-conjugated OX26 MAb was examined using a conjugate of 10 nm gold and an anti-mouse secondary antibody (Sigma). The PIN (10-30 μg) was incubated with undiluted IgG gold conjugate (5-15×10¹¹ gold particles) for 1 hour in 0.018 M Tris buffered saline, pH=8, with 0.9% bovine serum albumin, and 17% glycerol in a total volume of 58 μL, and then examined directly with electron microscopy.

Morphological evaluation was also performed for the pegylated nanoparticles without gold staining. Transmission electron microscopy was performed using a Jeol JEM-100CX II electron microscope at 80 kV. The nanoparticles were deposited on a 200 mesh formvar-coated copper grid followed by negative staining with 1-2% (w/v) phosphotungstic acid solution, pH=7. Negatives, taken at a 14,000 and 29,000 magnification, were scanned and enlarged in Adobe Photoshop 5.5 on a G4 Power Macintosh.

Dry weight content and turbidimetry measurements. The weight percent yield of the nanoparticle preparation method was calculated from the dry-weight content of the suspensions after nanoparticle washing with water. Three nanoparticle batches were prepared as described, except that after preparation nanoparticles were resuspended with 1 ml water. Half was used as standard nanoparticle suspension for calibrating the turbidimetry method, the other half was used for dry-weight content determination. The nanoparticles were diluted with 50 ml water, centrifuged at 45000 g and at 4° C. for 45 min and, after discarding the supernatant, was re-dispersed in 250 μl water. Nanoparticles were then dried at 80° C. for 16 h in a pre-weighed glass tube. The dry-weight was then measured and used to calculate the percent yield and the nanoparticle concentration of the standards.

Nanoparticle concentration was determined by turbidimetry measurements at 400 nm. The method was calibrated using the nanoparticle preparations the dry-weight contents of which were determined. The turbidity versus concentration response was linear over a concentration ranging from 0.05 to 1.00 mg/ml dry-weight content (r=0.9998, slope=0.9192, intercept=0.0014).

The synthesized methoxyPEG₂₆₀₀-PLA₄₀₀₀₀ and maleimide-PEG₃₅₀₀-PLA₄₀₀₀₀ copolymers had typical ¹H (FIGS. 4A and 4B) and ¹³C NMR (not shown) spectra and chemical shifts (Table 1) were in agreement with previously published data on PEG-PLA copolymers (17) and with NMR reference data available on maleimide functions. The number average molecular weights (Mn_{PLA}) of methoxyPEG₂₆₀₀-PLA₄₀₀₀₀ and of maleimide-PEG₃₅₀₀-PLA₄₀₀₀₀ as determined by ¹H NMR analysis are given in Table 2. Determined Mn_{PLA} for both copolymers were around 80% of values expected from the feed. For maleimide-PEG₃₅₀₀-PLA₄₀₀₀₀ the ratio of the peak area for maleimide-PEG₃₅₀₀ methylene protons versus the peak area for maleimide protons was 220, which was close to the ratio of 189 determined for the maleimide-PEG₃₅₀₀ used for the synthesis. This indicated that the maleimide function was mostly preserved in the final product.

TABLE 1
¹H and ¹³C NMR chemical shifts of protons and carbons of the two copolymers
synthesized. Signals from the methoxy end groups of the methoxyPEG-PLA copolymer,
from the α-methylene groups of PLA-connecting ethylene oxide units, from the
hydroxylated lactyl end units or from the carbons involved in the maleimide end
groups of the maleimide-PEG-PLA copolymer were not detected by the method.

| | Peak assignments | Chemical shifts δ (ppm) | | | |
|---|---|---|---|---|---|
| | on FIG. 2A and 2B | MethoxyPEG-PLA | | Maleimide-PEG-PLA | |
| Groups | (¹H NMR spectra) | ¹H NMR | ¹³C NMR | ¹H NMR | ¹³C NMR |
| CH₃^{a} | c | 1.56 (doublet) | 16.58 | 1.56 (doublet) | 16.56 |
| CH^{a} | b | 5.15 (quartet) | 68.95 | 5.14 (quartet) | 68.93 |
| C═O^{a} | | — | 169.51 | — | |
| CH₂^{b} | a | 3.63 (singlet) | 70.51 | 3.62 (singlet) | 70.49 |
| CH of maleimide | d | none | none | 6.69 (singlet) | too low |
| | | | | | signal |

^{a}from lactyl main units of PLA, ^{b}from ethylene oxide units of PEG

TABLE 2
Number average molecular weight data for the copolymers. Mn_{PEG} of
methoxyPEG and of Maleimide-PEG are provider's data, Mn_{PLA} were
determined by ¹H NMR.

| | Copolymer weight ratio (Mn_{PEG}:Mn_{PLA}) | |
|---|---|---|
| Copolymers | Theoretical (feed) | Determined by ¹H NMR |
| MethoxyPEG-PLA | 2600:46900 | 2600:37900 |
| Maleimide-PEG-PLA | 3501:45700 | 3501:36900 |

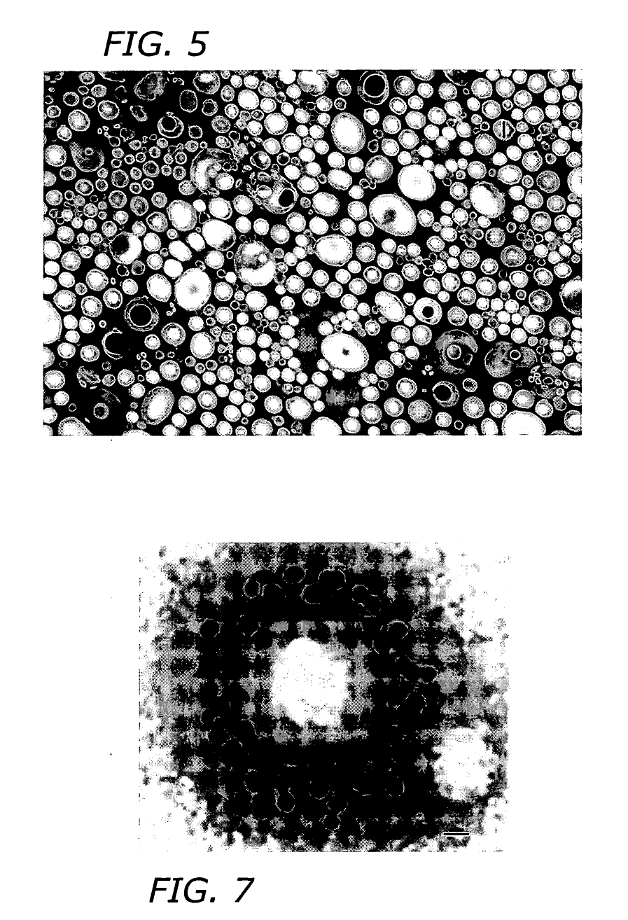

The pegylated nanoparticles were generally spherical and of regular size when examined by TEM (FIG. 5). Most pegylated nanoparticles were around 100 nm or below in diameter. A few were 200 to 300 nm in diameter. By QELS, the nanoparticle size distribution was monodisperse. The number-based nanoparticle diameter determined by QELS was 121±5 nm (n=3) with a standard deviation (describing the width of the nanoparticle size distribution) of 34±3 nm (n=3). The percent yield based on the dry weight content (17.8±0.8 mg/ml) determined by turbidimetry was calculated to be 63.7±4.9% (n=3).

FIG. 6 is an example of elution profile of the PINs obtained by Sepharose CL-4B gel filtration chromatography. Due to their sizes, the PINs eluted in fractions corresponding to the void volume of the column (from 8 to 11 ml), whereas the unconjugated OX26 MAb eluted in later fractions (from 17 to 32 ml). The average number of OX26 MAb's conjugated per nanoparticle of 121 nm in diameter was calculated for each fraction (Table 3), which gives a mean of 67±4 OX26 MAb per nanoparticle. FIG. 7 is a TEM where the OX26 MAb conjugated to the pegylated nanoparticle was visualized by binding to a conjugate of 10 nm gold and an anti-mouse IgG. The PIN is surrounded by a corona of gold particles owing to the presence of the OX26 MAb conjugated to the surface of the pegylated nanoparticles.

TABLE 3
Concentrations in immunonanoparticles and in OX26 MAb, and the
calculated average number of OX26 MAb per an average nanoparticle
of 121 nm. The values were determined in the fractions containing
immunonanoparticles and obtained by a Sepharose CL-4B gel filtration
chromatography

| | | nanoparticle | amount of | average number |
|---|---|---|---|---|
| Experiment | Fraction | amount | OX26 | of OX26 MAb |
| number | number^{a} | (mg)^{b} | MAb (nmol)^{c} | per nanoparticle |
| 1 | 9 | 2.8 | 0.31 | 65 |
| | 10 | 10.1 | 1.14 | 68 |
| | 11 | 5.3 | 0.64 | 71 |
| 2 | 9 | 1.9 | 0.18 | 60 |
| | 10 | 8.8 | 0.95 | 65 |
| | 11 | 3.4 | 0.36 | 64 |
| 3 | 8 | 0.5 | 0.06 | 76 |
| | 9 | 7.4 | 0.83 | 67 |
| | 10 | 5.4 | 0.60 | 67 |
| | 11 | 0.7 | 0.07 | 65 |

^{a}1 ml per fraction,
^{b}determined by turbidimetry,
^{c}determined by radioactivity measurements

The pegylated immunoparticles are designed to permit specific drug targeting to organs in vivo based on the receptor specificity of the targeting MAb. The immunonanoparticles may also be used in vitro for diagnostic purposes. The PINs were prepared with two copolymers: methoxyPEG-PLA, as the major non-functionalized component, and maleimide-PEG-PLA, as the minor functionalized copolymer to which can be conjugated any thiolated targeting ligand (FIGS. 3-4). The molecular weight (2600) of the methoxyPEG moiety was selected to form a PEG “corona” around the PLA nanoparticle core that inhibits the nanoparticle uptake by the RES, with an expected in vivo blood half-life of several hours (6). A higher molecular weight was chosen for the PEG spacer (3500) of the maleimide-PEG-PLA so that the maleimide function would protrude from the corona in order to be available for conjugation. Copolymers were synthesized by ring-opening polymerization of L-lactide, using stannous octoate as a catalyst. The polymerization initiators were methoxyPEG₂₆₀₀ or maleimide-PEG₃₅₀₀ in order to synthesize methoxyPEG₂₆₀₀-PLA and maleimide-PEG₃₅₀₀-PLA, respectively. Performed in an organic solvent (toluene or xylene), this method allows control of the length of the PLA chain and a low polydispersity (20). The NMR spectra confirmed the synthesis of both copolymers and permitted the calculation of the number average molecular weight (Mn) of the PLA moieties (FIGS. 4A and 4B, and Table 1). The maleimide protons are observed at δ 6.69 with a surface area ratio versus the PEG methylene proton (δ3.62) close to the ratio calculated for the maleimide-PEG₃₅₀₀. This result shows that the maleimide function was not altered during the synthesis. Molecular weights of the PLA moieties calculated from the ¹H-NMR spectra for both copolymers were almost equal and were 80% of the values expected from the L-lactide feed (Table 2).

In the preceding example, the immunonanoparticles were prepared with methoxyPEG₂₆₀₀-PLA₄₀₀₀₀ and maleimide-PEG₃₅₀₀-PLA₄₀₀₀₀ by the multiple emulsion/solvent evaporation method such that the inner aqueous phase can dissolve hydrophilic drugs and diagnostic agents to be entrapped within the nanoparticle core. Such drugs and diagnostic agents include a wide variety of small molecules, proteins, and nucleic acids (7, 9, 13 and 28). Sodium cholate was chosen as surfactant as previous work showed that cholate did not interfere with blood coagulation factors (21) and caused a reduction in both the hydrodynamic diameter and polydispersity index (5). Due to the hydrophilicity of PEG and to the hydrophobicity of PLA, resulting in a phase-separation of the two blocks in water, PEG moieties orient themselves towards the aqueous phase forming a “corona” layer around the PLA nanoparticle core (7 and 12). Some PEG chains may have an orientation toward the inner phase of the nanoparticles, when the multiple emulsion is prepared, and may be trapped within the inner aqueous phase during the hardening of the polymeric PLA core (13). The maleimide-PEG₃₅₀₀-PLA₄₀₀₀₀ was blended with methoxyPEG₂₆₀₀-PLA₄₀₀₀₀, according to a methoxyPEG:maleimide-PEG ratio of 1:30. This ratio was based on methods used for preparing pegylated immunoliposomes (2 and 22). Stable nanoparticles with satisfactory yields and concentrations were obtained with the pegylated nanoparticles having mean diameters of 121±5 nm and the nanoparticles appeared spherical by TEM (FIG. 5).

The procedure for antibody conjugation to the pegylated nanoparticle was derived from the method used to prepare pegylated immunoliposomes (2). The OX26 MAb, directed against the rat transferrin receptor, was chosen for the conjugation because prior work with PILs showed this MAb allowed for targeted drug delivery to transferrin receptor-rich organs such as brain of either small molecules (2 and 22) or non-viral plasmid DNA (23). The OX26 MAb was thiolated with 2-iminothiolane (2). A ratio of MAb:2-iminothiolane of 1:40 (mol/mol) was shown to provide the thiolation of an average of one primary amine per MAb (2). The thiolation of the OX26 MAb does not interfere with its target recognition (24). The OX26 MAb was conjugated to pegylated nanoparticles by formation of a thioether bond between the thiol groups of the MAb and the maleimide moiety at the distal end of maleimide-PEG₃₅₀₀-PLA₄₀₀₀₀. A reproducible coupling was achieved using a OX26 MAb:maleimide ratio of 1:3. Surface-linked OX26 MAb was detected by electron microscopy using a conjugate of anti IgG secondary antibody and 10 nm gold (FIG. 7). The number of OX26 MAb's conjugated per nanoparticle of 121 nm in diameter was calculated to be 67±4 (Table 3). This number should be sufficient for a wide variety of tissue targeting including brain targeting, since comparable degrees of MAb conjugation to pegylated liposomes with the OX26 MAb allows for effective drug or gene targeting to the brain (2 and 23).

In the preceding example of the present invention, a maleimide-PEG-PLA copolymer was synthesized from maleimide-PEG and lactide dimers by ring opening polymerization, without altering the maleimide function. This copolymer was blended with methoxyPEG-PLA to prepare functionalized pegylated PLA nanoparticles. A thiolated monoclonal antibody directed against the rat transferrin receptor was conjugated to these nanoparticles to obtain pegylated immunonanoparticles that are useful in providing targeted drug and diagnostic agent delivery of a variety of pharmaceuticals including small molecules, proteins, and gene medicines.

The maleimide-PEG-block-poly(α-hydroxy)acid functionalized compositions in accordance with the present invention permit the conjugation of any thiolated molecules to any material or device that incorporate them. The conjugation can be carried out on preformed materials in mild conditions that retain the biological activities of biomolecules. They therefore can have numerous therapeutic and/or biomedical applications: drug targeting, diagnostic purpose (in vitro or in clinics). Advantageously, the synthesis of the functionalized compositions does not necessitate particular equipment, and actual equipment designed to prepare poly(α-hydroxy)acid polymers or methoxyPEG-block-poly(α-hydroxy)acid copolymers can easily be adapted to prepare the functionalized compositions and immunonanoparticles in accordance with the present invention.

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the above preferred embodiments and examples, but is only limited by the following claims.

### Bibliography

1. D. Papahadjopoulos, T. M. Allen, A. Gabizon, E. Mayhew, K. Matthay, S. K. Huang, K. Lee, M. C. Woodle, D. D. Lasic, C. Redemann and F. J. Martin. Sterically stabilized liposomes: improvements in pharmacokinetics and antitumor therapeutic efficacy. Proc. Nat. Acad. Sci. U.S.A. 88: 11460-11464 (1991).
2. J. Huwyler, W. Dafang, and W. M. Pardridge. Brain drug delivery of small molecules using immunoliposomes. Proc. Natl. Acad. Sci. USA 93: 14164-14169 (1996).
3. J. M. Anderson and M. S. Shive. Biodegradation and biocompatibility of PLA and PLGA microspheres. Adv. Drug Deliv. Rev. 28: 5-24 (1997).
4. R. Gref, Y. Minamitake, M. T. Peracchia, V. Trubetskoy, V. Torchilin, and R. Langer. Biodegradable long-circulating nanospheres. Science 263: 1600-1603 (1994).
5. R. Gref, A. Domb, P. Quellec, T. Blunk, R. H. Muiller, J. M. Verbavatz, and R. Langer. The controlled intravenous delivery of drugs using PEG-coated sterically stabilized nanospheres. Adv. Drug Deliv. Rev. 16: 215-233 (1995).
6. T. Verrecchia, G. Spenlehauer, D. V. Bazile, A. Murry-Brelier, Y. Archimbaud, and M. Veillard. Non stealth (poly(lactic acid/albumin)) and Stealth PLA-PEG nanoparticles as injectable drug carriers. J. Control. Release. 36: 49-61 (1995).
7. M. T. Perracchia, R. Gref, Y. Minamitake, A. Domb, N. Lotan, and R. Langer. PEG-coated nanospheres from amphiphilic diblock and multiblock copomers: investigation of their drug encapsulation and release characteristics. J. Control. Release 46: 223-231 (1997).
8. M. F. Zambaux, F. Bonneaux, R. Gref, E. Dellacherie, and C. Vigneron. Protein C-loaded monomethoxypoly (ethylene oxide)-poly(lactic acid) nanoparticles. Int. J. Pharm. 212: 1-9 (2001).
9. C. Perez, A. Sanchez, D. Putnam, D. Ting, R. Langer, and M. J. Alonso. Poly(lactic acid)-poly(ethylene glycol) nanoparticles as new carriers for the delivery of plasmid DNA. J. Control. Release 75:211-224 (2001).
10. J. P. Plard and D. Bazile. Comparison of the safety profiles of PLA₅₀ and Me.PEG-PLA₅₀ nanoparticles after single dose intravenous administration to rat. Colloids Surfaces B Biointerfaces 16:173-183 (1999).
11. K. Yasugi, T. Nakamura, Y. Nagasaki, and K. Kataoka. Sugar-installed polymer micelles: synthesis and micellization of poly(ethylene glycol)-poly(D,L-lactide) block copolymers having sugar groups at the PEG chain end. Macromolecules 32: 8024-8032 (1999).
12. D. Bazile, C. Prud'homme, M. T. Bassoullet, M. Marlard, G. Spenlehauer, and M. Veillard. Stealth Me.PEG-PLA nanoparticles avoid uptake by the mononuclear phagocytes system. J. Pharm. Sci. 84: 493-498 (1995).
13. T. Riley, T. Govender, S. Stolnik, C. D. Xiong, M. C. Garnett, L. Illum, and S. S. Davis. Colloidal stability and drug incorporation aspects of micellar-like PLA-PEG nanoparticles. Colloids Surfaces B Biointerfaces, 16:147-159 (1999).
14. P. Quellec, R. Gref, L. Perrin, E. Dellacherie, F. Sommer, J. M. Verbavatz, M. J. Alonso. Protein encapsulation within polyethylene glycol-coated nanospheres. I. Physicochemical characterization. J. Biomed. Mater. Res. 42: 45-54 (1998).
15. Y.-S. Kang and W. M. Pardridge. Use of neutral avidin improves pharmacokinetics and brain delivery of biotin bound to an avidin-monoclonal antibody conjugate. J. Pharmacol. Exp. Ther. 269: 344-350 (1994).
16. W. M. Pardridge, J. L. Buciak, and T. Yoshikawa. Transport of recombinant CD4 through the rat blood-brain barrier in vivo. J. Pharmacol. Exp. Ther. 261: 1175-1180 (1992).
17. S. Tanodekaew, R. Pannu, F. Heatley, D. Attwood, and C. Booth. Association and surface properties of diblock copolymers of ethylene oxide and DL-lactide in aqueous solution. Macromol. Chem. Phys. 198: 927-944 (1997).
18. The Sadtler Handbook of Proton NMR Spectra. W. W. Simons, Ed. Sadtler Research Laboratories, Philadelphia, 1978.
19. Sadtler Standard Carbon-13 NMR Spectra. Sadtler Research Laboratories, Philadelphia.
20. P. Bouillot, A. Petit, and E. Dellacherie. Protein encapsulation in biodegradable amphiphilic microspheres I. Polymer synthesis and characterization, and microsphere elaboration. J. Appl. Pol. Sci. 68: 1695-1702 (1998).
21. H. Sahli, J. Tapon-Bretaudiere, A. M. Fischer, C. Sternberg, G. Spenlehauer, T. Verrecchia, and D. Labarre. Interactions of poly(lactic acid) and poly(lactic acid-co-ethylene oxide) nanoparticles with the plasma factors of the coagulation system. Biomaterials 18: 281-288 (1997).
22. J. Huwyler, J. Yang, and W. M. Pardridge. Receptor mediated delivery of daunomycin using immunoliposomes: pharmacokinetics and tissue distribution in the rat. J. Pharmacol. Exp. Ther. 282: 1541-1546 (1997).
23. N. Shi and W. M. Pardridge. Non invasive gene targeting to the brain. Proc. Natl. Acad. Sci. USA 97: 7567-7572 (2000).
24. W. M. Pardridge, R. J. Boado, and Y. S. Kang. Vector-mediated delivery of a polyamide (“peptide”) nucleic acid analogue through the blood-brain barrier in vivo. Proc. Natl. Acad. Sci. USA 92: 5592-5596 (1995).
25. Andreopoulos A. G., Hatzi E. and Doxastakis M. Synthesis and properties of poly(lactic acid). J Mater. Sci. Mater. Med. 10(1999)29-33
26. Gref R, Luck M, Quellec P, Marchand M, Dellacherie E, Harnisch S, Blunk T, Muller R H. ‘Stealth’ corona-core nanoparticles surface modified by polyethylene glycol (PEG): influences of the corona (PEG chain length and surface density) and of the core composition on phagocytic uptake and plasma protein adsorption. Colloids Surf B Biointerfaces 18(2000)301-313.
27. Stolnik S, Ilium L and Davis S S. Long circulating microparticulate drug carrier. Adv. Drug Deliv. Rev. 16(1995)195-214.
28. Zambaux M F, Bonneaux F, Gref R, Dellacherie E and Vigneron C. Preparation and characterization of protein C-loaded PLA nanoparticles. J. Control. Rel. 60(1999) 179-188.

1. A functionalized composition for use in forming an immunonanoparticle, said functionalized composition comprising:
a nanoparticle-forming polymer;
a polymeric strand having a first end attached to said nanoparticle-forming polymer and a second end;
a conjugation agent attached to the second end of said polymeric strand.

2. A functionalized moiety for use in forming an immunonanoparticle according to claim 1 that further includes a targeting agent attached to said conjugation agent.

3. A functionalized composition for use in forming an immunonanoparticle according to claim 1 wherein said nanoparticle forming polymer is selected from the group consisting of poly(lactic acid), poly(glycolide), poly(lactide-co-glycolide), polybutylcyanocrylate, poly(ε-caprolactone), monomethoxypoly(ethylene oxide)-poly(lactic acid), hexadecylcyanoacrylate, and copolymers thereof.

4. A functionalized composition for use in forming an immunonanoparticle according to claim 1 wherein said polymeric strand is selected from the group of polymers consisting of polyethylene glycol or sphingomyelin.

5. A functionalized composition for use in forming an immunonanoparticle according to claim 4 wherein said polymeric strand is polyethylene glycol that has a molecular weight of between 1000 and 50,000 Daltons.

6. A functionalized composition for use in forming an immunonanoparticle according to claim 1 wherein said conjugation agent is selected from the group consisting of a maleimide, hydroxysuccinimide, hydrazide, carboxylic acid, or a primary amine group.

7. A functionalized composition for use in forming an immunonanoparticle according to claim 5 wherein said conjugation agent is maleimide.

8. A functionalized composition for use in forming an immunonanoparticle according to claim 7 wherein said nanoparticle forming polymer is poly(lactic acid).

9. A nanoparticle composition comprising a plurality of functionalized compositions according to claim 1, said nanoparticle composition including a core comprising said nanoparticle-forming polymers and a corona surrounding said core that comprises said polymeric strands.

10. A nanoparticle composition according to claim 9 that comprises a plurality of non-functionalized compositions wherein each of said non-functionalized compositions comprises a nanoparticle-forming polymer and a polymeric strand having a first end attached to said nanoparticle-forming polymer and a second end and wherein said core includes nanoparticle-forming polymers from both said functionalized and non-functionalized compositions and wherein said corona includes polymeric strands from both said functionalized and non-functionalized compositions.

11. A nanoparticle composition according to claim 9 wherein the nanoparticle forming polymers of said functionalized compositions are selected from the group consisting of poly(lactic acid), poly(glycolide), poly(lactide-co-glycolide), polybutylcyanocrylate, poly(ε-caprolactone), monomethoxypoly(ethylene oxide)-poly(lactic acid), hexadecylcyanoacrylate, and copolymers thereof.

12. A nanoparticle composition according to claim 10 wherein the nanoparticle forming polymers of said non-functionalized compositions are selected from the group consisting of poly(lactic acid), poly(glycolide), poly(lactide-co-glycolide), polybutylcyanocrylate, poly(ε-caprolactone), monomethoxypoly(ethylene oxide)-poly(lactic acid), hexadecylcyanoacrylate, and copolymers thereof

13. A nanoparticle composition according to claim 9 wherein said polymeric strands of said functionalized compositions are selected from the group of polymers consisting of polyethylene glycol or sphingomyelin.

14. A nanoparticle composition according to claim 10 wherein said polymeric strands of said non-functionalized compositions are selected from the group of polymers consisting of polyethylene glycol or sphingomyelin.

15. A nanoparticle composition according to claim 13 wherein said polymeric strands comprise polyethylene glycol that has a molecular weight of between 1000 and 50,000 Daltons.

16. A nanoparticle composition according to claim 14 wherein said polymeric strands comprise polyethylene glycol that has a molecular weight of between 1000 and 50,000 Daltons.

17. A nanoparticle composition according to claim 15 wherein said polymeric strands of said non-functionalized compositions comprise polyethylene glycol that has a molecular weight of between 1000 and 50,000 Daltons.

18. A nanoparticle composition according to claim 10 wherein the molecular weight of the polymeric strands in said functionalized compositions is greater than the molecular weight of the polymeric strands in said non-functionalized compositions.

19. A nanoparticle composition according to claim 17 wherein the molecular weight of the polyethylene glycol strands in said functionalized compositions is greater than the molecular weight of the polyethylene glycol strands in said non-functionalized compositions

20. A nanoparticle composition according to claim 9 wherein said conjugation agent is selected from the group consisting of a maleimide, hydroxysuccinimide, hydrazide, carboxylic acid, or a primary amine group.

21. A nanoparticle composition according to claim 20 wherein said conjugation agent is maleimide.

22. A nanoparticle composition according to claim 9 wherein said nanoparticle forming polymer is poly(lactic acid).

23. A nanoparticle according to claim 9 wherein the size of said immunonanoparticle precursor ranges from 50 to 150 nanometers.

24. A nanoparticle composition comprising a plurality of functionalized compositions according to claim 2, said nanoparticle composition including a core comprising said nanoparticle-forming polymers and a corona surrounding said core that comprises said polymeric strands.

25. A nanoparticle composition according to claim 24 that comprises a plurality of non-functionalized compositions wherein each of said non-functionalized compositions comprises a nanoparticle-forming polymer and a polymeric strand having a first end attached to said nanoparticle-forming polymer and a second end and wherein said core includes nanoparticle-forming polymers from both said functionalized and non-functionalized compositions and wherein said corona includes polymeric strands from both said functionalized and non-functionalized compositions.

26. A nanoparticle composition according to claim 24 wherein the nanoparticle forming polymers of said functionalized compositions are selected from the group consisting of poly(lactic acid), poly(glycolide), poly(lactide-co-glycolide), polybutylcyanocrylate, poly(ε-caprolactone), monomethoxypoly(ethylene oxide)-poly(lactic acid), hexadecylcyanoacrylate, and copolymers thereof.

27. A nanoparticle composition according to claim 25 wherein the nanoparticle forming polymers of said non-functionalized compositions are selected from the group consisting of poly(lactic acid), poly(glycolide), poly(lactide-co-glycolide), polybutylcyanocrylate, poly(ε-caprolactone), monomethoxypoly(ethylene oxide)-poly(lactic acid), hexadecylcyanoacrylate, and copolymers thereof.

28. A nanoparticle composition according to claim 24 wherein said polymeric strands of said functionalized compositions are selected from the group of polymers consisting of polyethylene glycol or sphingomyelin.

29. A nanoparticle composition according to claim 25 wherein said polymeric strands of said non-functionalized compositions are selected from the group of polymers consisting of polyethylene glycol or sphingomyelin.

30. A nanoparticle composition according to claim 28 wherein said polymeric strands comprise polyethylene glycol that has a molecular weight of between 1000 and 50,000 Daltons.

31. A nanoparticle composition according to claim 29 wherein said polymeric strands comprise polyethylene glycol that has a molecular weight of between 1000 and 50,000 Daltons.

32. A nanoparticle composition according to claim 30 wherein said polymeric strands of said non-functionalized compositions comprise polyethylene glycol that has a molecular weight of between 1000 and 50,000 Daltons.

33. A nanoparticle composition according to claim 29 wherein the molecular weight of the polymeric strands in said functionalized compositions is greater than the molecular weight of the polymeric strands in said non-functionalized compositions.

34. A nanoparticle composition according to claim 32 wherein the molecular weight of the polyethylene glycol strands in said functionalized compositions is greater than the molecular weight of the polyethylene glycol strands in said non-functionalized compositions

35. A nanoparticle composition according to claim 24 wherein said conjugation agent is selected from the group consisting of a maleimide, hydroxysuccinimide, hydrazide, carboxylic acid, or a primary amine group.

36. A nanoparticle composition according to claim 35 wherein said conjugation agent is maleimide.

37. A nanoparticle composition according to claim 24 wherein said nanoparticle forming polymer is poly(lactic acid).

38. A nanoparticle composition according to claim 24 wherein the size of said immunonanoparticle precursor ranges from 50 to 150 nanometers.

39. A nanoparticle composition according to claim 10 wherein the ratio of functionalized compositions to non-functionalized compositions in said immunonanoparticle precursor is between 1:5 and 1:100.

40. A nanoparticle composition according to claim 25 wherein the ratio of functionalized compositions to non-functionalized compositions in said immunonanoparticle is between 1:5 and 1:100.

41. A nanoparticle composition according to claims 9-23 and 39 wherein said core includes a drug or diagnostic agent.

42. A nanoparticle composition according to claims 24-38 and 40 wherein said core includes a drug or diagnostic agent.

43. A method for making a nanoparticle composition comprising the step of combining a plurality of said functionalized compositions according to claim 1 to form an nanoparticle composition that includes a core comprising said nanoparticle-forming polymers and a corona surrounding said core that comprises said polymeric strands and treating said nanoparticle with a targeting agent that binds to said conjugation agents to form said nanoparticle composition.

44. A method for making a nanoparticle composition comprising the step of combining a plurality of said functionalized compositions according to claim 2 to form said nanoparticle composition that includes a core comprising said nanoparticle-forming polymers, a corona surrounding said core that comprises said polymeric strands and a plurality of targeting agents that are bound to said conjugation agents.

45. A method for making a nanoparticle composition according to claim 43 which comprises the step of combining a non-functionalized composition with said functionalized composition, said non-functionalized composition comprising a nanoparticle-forming polymer and a polymeric strand having a first end attached to said nanoparticle-forming polymer and a second end.

46. A method for making a nanoparticle composition according to claim 44 which comprises the step of combining a non-functionalized composition with said functionalized composition, said non-functionalized composition comprising a nanoparticle-forming polymer and a polymeric strand having a first end attached to said nanoparticle-forming polymer and a second end.

47. A method for making a nanoparticle composition according to claim 45 wherein the ration of functionalized compositions to non-functionalized compositions that are combined together to form said immunonanoparticle is between 1:5 and 1:100.

48. A method for making a nanoparticle composition according to claim 46 wherein the ratio of functionalized compositions to non-functionalized compositions that are combined together to form said immunonanoparticle is between 1:5 and 1:100.

49. A method for making a nanoparticle composition according to claim 43 that includes the additional step of incorporating a drug or diagnostic agent into the core of said immunonanoparticle.

50. A method for making a nanoparticle composition according to claim 44 that includes the additional step of incorporating a drug or diagnostic agent into the core of said immunonanoparticle.
